(19) United States
(12) Patent Application Publication
(10) Pub. No.: US 2009/0239917 A1
(43) Pub. Date: Sep. 24, 2009

(54) Heterocyclic Cycloalkyl Compounds, a Process for their Preparation and Pharmaceutical Compositions Containing Them

(75) Inventors: Aurelie Hurtevent, Lille (FR); Jean-Martial L'Helgoual'ch, Lille (FR); Pascal Carato, Lille (FR); Nicolas Lebegue, La Bassee (FR); Veronique Leclerc, Avelin (FR); Morgan Le Naour, Lille (FR); Pascal Berthelot, Haubourdin (FR); Catherine Dacquet, Villeneuve d'Ascq (FR); Alain Ktorza, Nogent Sur Marne (FR); Daniel-Henri Caignard, Boisemont (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: Les Laboratoires Servier, Courbevoie Cedex (FR)

(21) Appl. No.: 12/224,861

(22) PCT Filed: Mar. 5, 2007
(86) PCT No.: PCT/FR2007/000384

§ 371 Date: Nov. 20, 2008

(30) Foreign Application Priority Data
Mar. 6, 2006 (FR) 0601954

(51) Int. Cl. A61K 31/428; C07D 277/68; A61P 3/04; A61P 3/10; A61P 9/10; A61P 25/28; A61P 19/10; A61P 35/00

(52) U.S. Cl. 514/367; 548/170; 548/169; 548/171

(57) ABSTRACT

Compounds of formula (I):

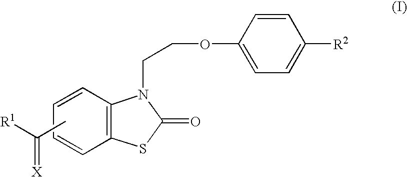

wherein:
R¹ represents a (C₃-C₈)cycloalkyl group,
R² represents a group of formula (II) as defined in the description,
X represents an oxygen atom or an N—OR′ group wherein R′ represents a hydrogen atom, a linear or branched (C₁-C₆)alkyl group, an aryl group or an aryl-(C₁-C₆)alkyl group in which the alkyl moiety may be linear or branched.

Medicinal products containing the same which are useful as hypoglycaemic and hypolipaemic agents.

The present invention relates to new heterocyclic cycloalkyl compounds, to a process for their preparation and to pharmaceutical compositions containing them.

The compounds described in the present invention are new and have pharmacological properties that are of special interest: they are excellent hypoglycaemic and hypolipaemic agents.

The treatment of non-insulin-dependent type II diabetes remains unsatisfactory despite the introduction onto the market of a large number of oral hypoglycaemic compounds intended to facilitate the secretion of insulin and to promote its action in peripheral target tissues.

During the last ten years, a class of compounds having a thiazolidinedione structure (U.S. Pat. No. 5,089,514, U.S. Pat. No. 5,306,726) has demonstrated a marked anti-diabetic activity by promoting sensitivity to insulin in the target peripheral tissues (skeletal muscle, liver, adipose tissue) of animal models having non-insulin-dependent type II diabetes. Those compounds also lower the levels of insulin and levels of lipids in the same animal models and induce in vitro differentiation of preadipocyte cell lines into adipocyte cell lines (A. Hiragun et al., J. Cell. Physiol., 1988, 134, 124-130; R. F. Kleitzen et al., Mol. Pharmacol., 1992, 41, 393-398).

The treatment of preadipocyte cell lines with the thiazolidinedione rosiglitazone brings about inducement of the expression of specific genes of the lipid metabolism, such as aP2 and adipsin, and also the expression of the glucose transporters GLUT1 and GLUT4, suggesting that the effect of the thiazolidinediones observed in vivo may be mediated via adipose tissue. That specific effect is obtained by the stimulation of nuclear transcription factors: <<peroxisome proliferator-activated receptor gamma >> (PPAR γ2). Such compounds are capable of restoring sensitivity to insulin in peripheral tissues, such as adipose tissue or skeletal muscle (J. E. Gerich, New Engl. Med., 19, 321, 1231-1245).

Compounds having a thiazolidinedione structure (troglitazone, rosiglitazone) have demonstrated disturbing side effects in man, however, especially liver problems (Script No 2470, 1999, Sep. 8^{th}, 25).

A large number of hypoglycaemic agents have significant side effects (hepatic, cardiac, haematopoietic), which limit their long-term use in the treatment of non-insulin-dependent type II diabetes.

The development of new therapeutic agents that are less toxic and that are active over the long term is absolutely necessary in this pathology.

Moreover, hyperlipidaemia is often observed in diabetics (Diabetes Care, 1995, 18 (supplement 1), 86/8/93). The association of hyperglycaemia with hyperlipidaemia increases the risk of cardiovascular disease in diabetics. Hyperglycaemia, hyperlipidaemia and obesity have become pathologies of the modern world marked by the intake of food in large quantities and a chronic lack of exercise.

The increase in frequency of those pathologies calls for the development of new therapeutic agents that are active in such disorders: compounds having an excellent hypoglycaemic and hypolipaemic activity whilst avoiding the side effects observed with thiazolidinediones are consequently very beneficial in the treatment and/or prophylaxis of those pathologies, and are indicated especially in the treatment of non-insulin-dependent type II diabetes for reducing peripheral insulin resistance and for normalising glucose control.

In addition to the fact that they are new, the compounds of the present invention meet the above pharmacological criteria and are excellent hypoglycaemic and hypolipaemic agents.

The present invention relates more especially to compounds of formula (I):

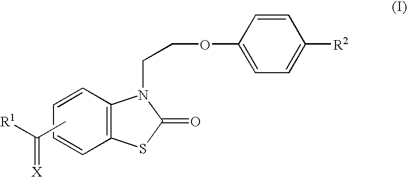

wherein:
R¹ represents a (C₃-C₈)cycloalkyl group,
R² represents a group of formula (II):

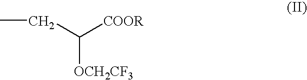

wherein R represents a hydrogen atom or a linear or branched (C₁-C₆)alkyl group,
X represents an oxygen atom or an N—OR′ group wherein R′ represents a hydrogen atom, a linear or branched (C₁-C₆)alkyl group, an aryl group or an aryl-(C₁-C₆)alkyl group in which the alkyl moiety may be linear or branched,
   to their geometric isomers, enantiomers and diastereoisomers, and also to the pharmaceutically acceptable addition salts thereof with an acid or a base,
   wherein:
“geometric isomers” is understood to mean that, when X represents an N—OR′ group, the oxime R¹—C(═N—OR′)— may have the Z or E configuration,
“aryl” is understood to mean a phenyl or naphthyl group, wherein those groups may optionally be substituted by from 1 to 3 groups selected from linear or branched (C₁-C₆)-alkyl, linear or branched (C₁-C₆)polyhaloalkyl, linear or branched (C₁-C₆)alkoxy, hydroxy, carboxy, formyl, amino (optionally substituted by one or two linear or branched (C₁-C₆)-alkyl groups), ester, amido, nitro, cyano, and halogen atoms.

Amongst the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, methanesulphonic acid, camphoric acid, oxalic acid, etc. . . .

Amongst the pharmaceutically acceptable bases there may be mentioned, without implying any limitation, sodium hydroxide, potassium hydroxide, triethylamine, tert-butylamine, etc.

Preferred compounds of the invention are compounds of formula (I) wherein R¹ represents a cyclopropyl group.

R preferably represents a hydrogen atom or an ethyl group.

The preferred R² group of the compounds of formula (I) according to the invention is the group —CH₂—CH(OCH₂CF₃)(COOH).

Advantageously, the R² group has the R or S configuration.

X preferably represents an oxygen atom.

Compounds of formula (I) according to the invention wherein R represents a hydrogen atom are advantageously converted into salts, and more especially into sodium salts.

Even more especially, the invention relates to the following compounds of formula (I):
ethyl 3-{4-[2-(6-(cyclopropylcarbonyl)-2-oxo-1,3-benzothiazol-3(2H)-yl)ethoxy]phenyl}-2-(2,2,2-trifluoroethoxy)propanoate,
3-{4-[2-(6-(cyclopropylcarbonyl)-2-oxo-1,3-benzothiazol-3(2H)-yl)ethoxy]phenyl}-2-(2,2,2-trifluoroethoxy)propanoic acid,
ethyl 3-{4-[2-(6-[cyclopropyl(hydroxyimino)methyl]-2-oxo-1,3-benzothiazol-3(2H)-yl)-ethoxy]phenyl}-2-(2,2,2-trifluoroethoxy)propanoate,
3-{4-[2-(6-[cyclopropyl(hydroxyimino)methyl]-2-oxo-1,3-benzothiazol-3 (2H)-yl)-ethoxy]phenyl}-2-(2,2,2-trifluoroethoxy)propanoic acid,
ethyl 3-{4-[2-(6-[cyclopropyl(methoxyimino)methyl]-2-oxo-1,3-benzothiazol-3(2H)-yl)ethoxy]phenyl}-2-(2,2,2-trifluoroethoxy)propanoate,
3-{4-[2-(6-[cyclopropyl(methoxyimino)methyl]-2-oxo-1,3-benzothiazol-3(2H)-yl)-ethoxy]phenyl}-2-(2,2,2-trifluoroethoxy)propanoic acid,
(S)-3-{4-[2-(6-(cyclopropylcarbonyl)-2-oxo-1,3-benzothiazol-3(2H)-yl)ethoxy]phenyl}-2-(2,2,2-trifluoroethoxy)propanoic acid,
(R)-3-{4-[2-(6-(cyclopropylcarbonyl)-2-oxo-1,3-benzothiazol-3(2H)-yl)ethoxy]phenyl}-2-(2,2,2-trifluoroethoxy)propanoic acid,
sodium (S)-3-{4-[2-(6-(cyclopropylcarbonyl)-2-oxo-1,3-benzothiazol-3(2H)-yl)ethoxy]-phenyl}-2-(2,2,2-trifluoroethoxy)propanoate,
sodium (R)-3-{4-[2-(6-(cyclopropylcarbonyl)-2-oxo-1,3-benzothiazol-3(2H)-yl)ethoxy]-phenyl}-2-(2,2,2-trifluoroethoxy)propanoate.
potassium (S)-3-{4-[2-(6-(cyclopropylcarbonyl)-2-oxo-1,3-benzothiazol-3(2H)-yl)ethoxy]phenyl}-2-(2,2,2-trifluoroethoxy)propanoate,
potassium (R)-3-{4-[2-(6-(cyclopropylcarbonyl)-2-oxo-1,3-benzothiazol-3(2H)-yl)ethoxy]phenyl}-2-(2,2,2-trifluoroethoxy)propanoate.

The enantiomers, diastereoisomers, and also pharmaceutically acceptable addition salts with an acid or a base of the preferred compounds of the invention form an integral part of the invention.

The present invention relates also to a process for the preparation of the compounds of formula (I) which is characterised in that there is used as starting material a compound of formula (III):

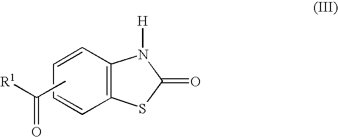

wherein R¹ is as defined for formula (I),
which is condensed in basic medium with a compound of formula (IV):

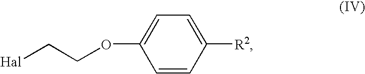

wherein R² is as defined for formula (I) and Hal represents a halogen atom, to yield a compound of formula (I/a), a particular case of the compounds of formula (I):

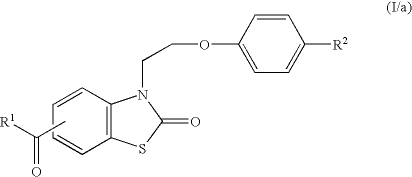

wherein R¹ and R² are as defined for formula (I),
which is optionally subjected to the action of a compound of formula R′O—NH₂, wherein R′ is as defined for formula (I), to yield a compound of formula (I/b), a particular case of the compounds of formula (I):

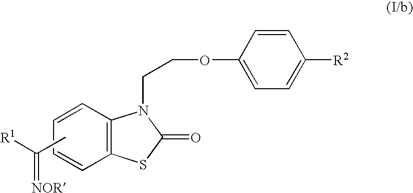

wherein R¹, R² and R′ are as defined for formula (I),
which compounds of formulae (I/a) and (I/b) may be purified according to a conventional purification technique, are converted, if desired, into addition salts with a pharmaceutically acceptable acid or base, and are optionally separated into isomers according to a conventional separation technique.

An advantageous variant relates to a process for the preparation of the compounds of formula (I/b) which is characterised in that there is used as starting material a compound of formula (III):

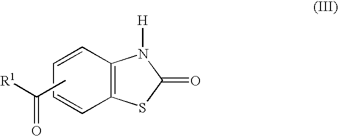

wherein R¹ is as defined for formula (I),
which is condensed with a compound of formula R′O—NH₂, wherein R′ is as defined for formula (I), to yield a compound of formula (V):

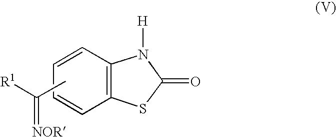

wherein R¹ and R′ are as defined for formula (I),
which is condensed in basic medium with a compound of formula (IV):

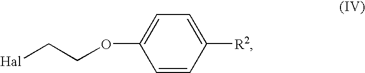

wherein R² is as defined for formula (I) and Hal represents a halogen atom, to yield a compound of formula (I/b), a particular case of the compounds of formula (I):

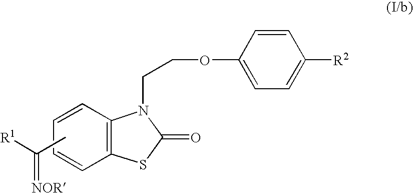

wherein R¹, R² and R′ are as defined for formula (I),
which may be purified according to a conventional purification technique, is converted, if desired, into addition salts with a pharmaceutically acceptable acid or base, and is optionally separated into isomers according to a conventional separation technique.

The compounds of formula (III) are commercial products or are readily obtainable by the person skilled in the art by conventional chemical reactions or reactions described in the literature.

The present invention relates also to a process for the preparation of compounds of formula (I) wherein the group R² has the R or S configuration, which is characterised in that there is used as starting material a compound of formula (I/c), a particular case of the compounds of formula (I):

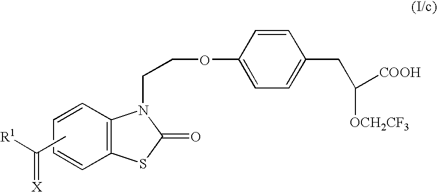

wherein X and R¹ are as defined for formula (I),
which is condensed, in the presence of a coupling agent, (S)-phenylglycinol, to yield a compound of formula (VI):

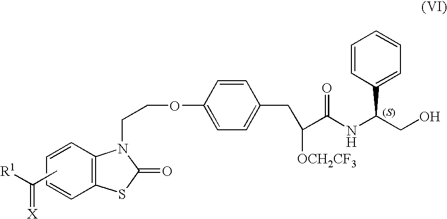

wherein X and R¹ are as defined hereinbefore,
which is purified by chromatography to yield compounds of formulae (VIIa) and (VIIb)

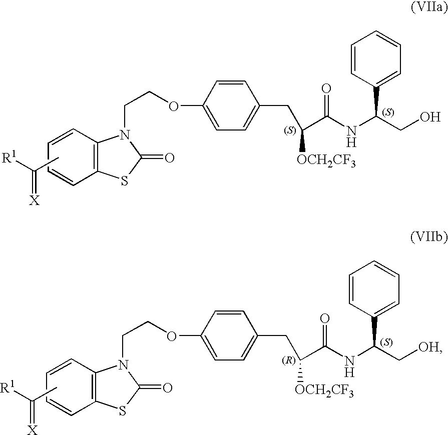

wherein X and R¹ are as defined hereinbefore,
which compounds of formulae (VIIa) and (VIIb) are subjected to hydrolysis in acid medium to yield compounds of formulae (I/d(a)) and (I/d(b)), particular cases of the compounds of formula (I):

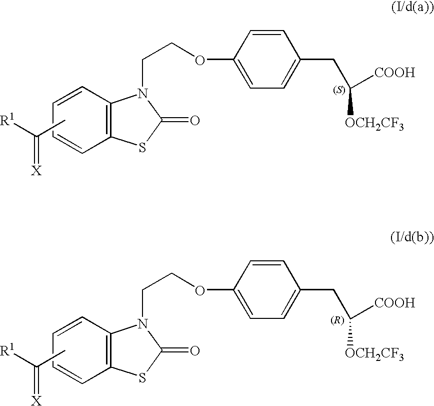

wherein X and R¹ are as defined hereinbefore,
which compounds of formulae (I/d(a)) and (I/d(b)) are optionally condensed with a compound of formula RₐOH, wherein Rₐ represents a linear or branched (C₁-C₆)alkyl group, to yield compounds of formulae (I/e(a)) and (I/e(b)), particular cases of the compounds of formula (I):

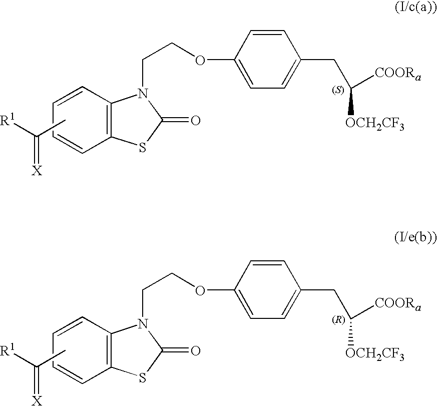

wherein X, Rₐ and R¹ are as defined hereinbefore,
or which compounds of formulae (I/d(a)) and (I/d(b)) are optionally subjected to the action of R_{b}OH, wherein R_{b} represents Na or K, to yield compounds of formulae (I/f(a)) and (I/f(b)), particular cases of the compounds of formula (I):

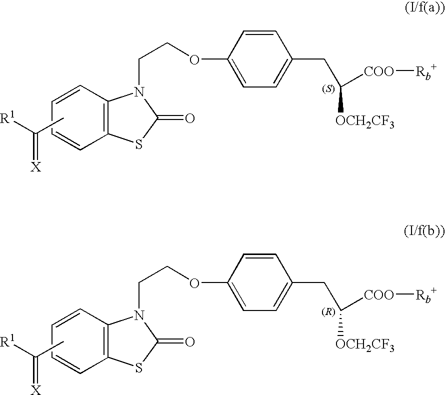

wherein X, R_{b} and R¹ are as defined hereinbefore,
the compounds of formulae (I/d(a)), (I/d(b)), (I/e(a)), (I/e(b)), (I/f(a)) and (I/f(b)) being purified according to a conventional purification technique.

The compounds of the present invention have very valuable pharmacological properties.

The compounds demonstrate especially an excellent activity in lowering blood glucose levels. As a result of such properties they can be used therapeutically in the treatment and/or prophylaxis of hyperglycaemia, dyslipidaemia and, more especially, in the treatment of non-insulin-dependent type II diabetes, glucose intolerance, disorders associated with syndrome X (including hypertension, obesity, insulin resistance, atherosclerosis, hyperlipidaemia), coronary artery disease and other cardiovascular diseases (including arterial hypertension, cardiac insufficiency, venous insufficiency), renal disorders (including glomerulonephritis, glomerulosclerosis, nephrotic syndrome, hypertensive nephrosclerosis), retinopathy, disorders associated with the activation of endothelial cells, psoriasis, polycystic ovary syndrome, dementia, diabetic complications and osteoporosis.

They can be used as aldose reductase inhibitors, for improving cognitive functions in dementia and for the complications of diabetes, intestinal inflammatory disorders, myotonic dystrophy, pancreatitis, arteriosclerosis, xanthoma.

The activity of these compounds is also recommended for the treatment and/or prophylaxis of other diseases, including type I diabetes, hypertriglyceridaemia, syndrome X, insulin resistance, dyslipidaemia in diabetics, hyperlipidaemia, hypercholesterolaemia, arterial hypertension, cardiac insufficiency, and cardiovascular disease, especially atherosclerosis.

The compounds are furthermore indicated for use in the regulation of appetite, especially in the regulation of food intake in subjects suffering from disorders such as obesity, anorexia, bulimia and anorexia nervosa.

The compounds can accordingly be used in the prevention or treatment of hypercholesterolaemia, obesity with advantageous effects on hyperlipidaemia, hyperglycaemia, osteoporosis, glucose intolerance, insulin resistance or disorders in which insulin resistance is a secondary physiopathological mechanism.

The use of those compounds enables reduction of total cholesterol, body weight, leptin resistance, plasma glucose, triglycerides, LDLs, VLDLs and also plasma free fatty acids.

The compounds can be used in association with HMG CoA reductase inhibitors, fibrates, nicotinic acid, cholestyramine, colestipol, probucol, GLP1, metformin, the biguanides or glucose reabsorption inhibitors and can be administered together or at different times to act in synergy in the patient treated.

They furthermore exhibit activity in cancer pathologies and especially hormone-dependent cancers, such as breast cancer and colon cancer, and also have an inhibiting effect on the angiogenesis processes implicated in those pathologies.

Amongst the pharmaceutical compositions according to the invention there may mentioned more especially those which are suitable for oral, parenteral, nasal, per- or trans-cutaneous, rectal, perlingual, ocular or respiratory administration and especially tablets or dragées, sublingual tablets, sachets, paquets, capsules, glossettes, lozenges, suppositories, creams, ointments, dermal gels and drinkable or injectable ampoules.

The dosage varies in accordance with the sex, age and weight of the patient, the administration route, the nature of the therapeutic indication or of any associated treatments and ranges from 0.1 mg to 1 g per 24 hours taken in 1 or more administrations.

The present invention relates also to a new association between a heterocyclic compound of formula (I) as defined hereinbefore and an antioxidant agent for obtaining pharmaceutical compositions for use in the treatment and/or prevention of obesity and overweight characterised by a body mass index greater than 25.

The antioxidant agents of the association according to the invention are, more especially, anti-free radical agents or free-radical trapping agents, antilipoperoxidant agents, chelating agents or agents capable of regenerating endogenous antioxidants such as glutathione, vitamin C or vitamin E, and also addition salts thereof with a pharmaceutically acceptable acid or base.

The antioxidant agent of the association according to the invention is more preferably represented by quinone compounds such as ubiquinone or coenzyme Q₁₀, which acts as a free-radical trapping agent but which is also capable of regenerating vitamin E.

The preferred association according to the invention is 3-{4-[2-(6-(cyclopropylcarbonyl)-2-oxo-1,3-benzothiazol-3(2H)-yl)ethoxy]phenyl}-2-(2,2,2-trifluoroethoxy)propanoic acid, its enantiomers and also its addition salts with a pharmaceutically acceptable base, and coenzyme Q₁₀.

Furthermore, the association according to the invention has entirely surprising pharmacological properties: the Applicant has demonstrated that a synergy exists between the two compounds of the association allowing a very significant reduction in body fat to be obtained, making it useful in the treatment and/or prevention of obesity and of overweight characterised by a body mass index greater than 25.

In the United States, obesity affects 20% of men and 25% of women. Patients having a body mass index (BMI=weight (kg)/height² (m²)) greater than or equal to 30 are considered to be obese (Int. J. Obes., 1998, 22, 39-47; Obesity Lancet, 1997, 350, 423-426). Obesity (BMI÷30) and overweight (25<BMI<30) can have various origins: they may come about following deregulation of food intake, following hormonal disturbance, or following administration of a treatment: treating type II diabetes with sulphonylureas causes patients to gain weight. Similarly, in type I (insulin-dependent) diabetes, insulin therapy is also a cause of weight gain in patients (In Progress in Obesity Research, 8^{th} International Congress on Obesity, 1999, 739-746; Annals of Internal Medicine, 1998, 128, 165-175).

Obesity and overweight are well-established risk factors for cardiovascular diseases: they are associated with a significant increase in the risk of cerebro-vascular accidents and non-insulin-dependent diabetes, because they predispose to insulin resistance, to dyslipidaemia and to the appearance of macrovascular disorders (nephropathy, retinopathy, angiopathy).

Further pathologies are the consequence of obesity or overweight: there may be mentioned, in particular, vesicular calculi, respiratory dysfunction, several forms of cancer and, in the case of very severe obesity, premature death (N. Engl. J. Med., 1995, 333, 677-385; JAMA, 1993, 270, 2207-2212).

The association according to the invention allows a weight loss to be obtained which, even if moderate, significantly reduces all the risk factors associated with obesity (Int. J. Obes., 1997, 21, 55-9; Int. J. Obes., 1992, 21, S5-9).

The association according to the invention will therefore be found to be useful in the treatment and/or prevention of obesity and of overweight characterised by a body mass index greater than 25.

The invention accordingly relates to the use of the association between a compound of formula (I) and an antioxidant agent in obtaining pharmaceutical compositions intended for the treatment and/or prevention of obesity and of overweight characterised by a body mass index greater than 25 and less than 30.

More especially, the association according to the invention is useful in the treatment and/or prevention of obesity and of overweight characterised by a body mass index greater than 25 and less than 30 induced by therapeutic treatment, such as treatment of type I or type II diabetes.

The invention accordingly relates to the use of the association between a compound of formula (I) and an antioxidant agent in obtaining pharmaceutical compositions intended for the treatment and/or prevention of obesity and of overweight characterised by a body mass index greater than 25 and less than 30 induced by therapeutic treatment, such as treatment of type I or type II diabetes.

The invention relates also to pharmaceutical compositions comprising the association between a compound of formula (I) and an antioxidant agent, as defined hereinbefore, in combination with one or more pharmaceutically acceptable excipients.

Among the pharmaceutical compositions according to the invention there may be mentioned, more especially, those that are suitable for oral, parenteral or nasal administration, tablets or dragées, sublingual tablets, capsules, lozenges, suppositories, creams, ointments, dermal gels, etc.

More especially, the invention relates to pharmaceutical compositions comprising a compound of formula (I) as defined hereinbefore and an antioxidant agent, such as coenzyme Q₁₀ or vitamin E, in combination with one or more pharmaceutically acceptable excipients.

The dosage used varies according to the sex, age and weight of the patient, the administration route, the nature of the therapeutic indication or of any associated treatments and ranges from 0.1 mg to 1 g of each component of the association per 24 hours in one or more administrations.

The Examples which follow illustrate the invention but do not limit it in any way.

### EXAMPLE 1

### Ethyl 3-{4-[2-(6-(cyclopropylcarbonyl)-2-oxo-1,3-benzothiazol-3(2H)-yl)ethoxy]phenyl}-2-(2,2,2-trifluoroethoxy)propanoate

Add potassium carbonate (0.01044 mol) and then 6-(cyclopropylcarbonyl)-1,3-benzothiazol-2(3H)-one (0.00453 mol) to 20 ml of dimethylformamide. Heat at 100° C. for one hour. Add ethyl 3-[4-(2-chloroethoxy)phenyl]-2-(2,2,2-trifluoroethoxy)propanoate (0.00348 mol) and heat at 150° C. for 16 hours. Evaporate off the dimethylformamide. Take up the residue in 50 ml of water and then extract twice with 50 ml of dichloromethane each time. The organic phase is dried over MgSO₄ and then evaporated. The residue is recrystallised from methanol and yields the title product in the form of a white powder.

Melting point: 113-115° C.

### EXAMPLE 2

### 3-{4-[2-(6-(Cyclopropylcarbonyl)-2-oxo-1,3-benzothiazol-3(2H)-yl)-ethoxy]phenyl}-2-(2,2,2-trifluoroethoxy)propanoic acid

0.9 g of the compound obtained in Example 1 is dissolved in a tetrahydrofuran/water (12/8 ml) mixture. 0.12 g of lithium hydroxide in a minimum of water is then added. The reaction mixture is stirred at 50° C. for one night. The tetrahydrofuran is then evaporated off, and the solution is hydrolysed and then acidified with 3N HCl. The reaction mixture is extracted with ethyl acetate, and the organic phase is dried over magnesium sulphate, filtered and then evaporated under reduced pressure. The residue obtained is purified on silica gel using a dichloromethane/methanol (95/5) eluant to yield the title product in the form of a white powder.

Melting point: 124-128° C.

### Elemental Microanalysis:

| | C % | H % | N % |
|---|---|---|---|
| Calculated: | 56.58 | 4.35 | 2.75 |
| Found: | 56.53 | 4.31 | 2.44 |

### EXAMPLE 3

### Ethyl 3-{4-[2-(6-[cyclopropyl(hydroxyimino)methyl]-2-oxo-1,3-benzothiazol-3(2H)-yl)ethoxy]phenyl}-2-(2,2,2-trifluoroethoxy)-propanoate

Dissolve the compound obtained in Example 1 (0.00198 mol), hydroxylamine hydrochloride (0.00594 mol) and pyridine (0.00594 mol) in 30 ml of methanol. Heat the solution at reflux with mechanical stirring for 5 hours. Evaporate to dryness, hydrolyse the residue in 100 ml of water, and acidify with 1N hydrochloric acid to pH 1. Suction-filter off and wash the precipitate obtained with petroleum ether. Purify on silica gel using an ethyl acetate/cyclohexane (3/7) eluant to yield the title product in the form of a white powder.

Melting point: 105-107° C.

### Elemental Microanalysis:

| | C % | H % | N % |
|---|---|---|---|
| Calculated: | 56.52 | 4.93 | 5.07 |
| Found: | 56.53 | 5.31 | 5.34 |

### EXAMPLE 4

### 3-{4-[2-(6-[Cyclopropyl(hydroxyimino)methyl]-2-oxo-1,3-benzothiazol-3(2H)-yl)ethoxy]phenyl}-2-(2,2,2-trifluoroethoxy)propanoic acid

The procedure is as in Example 2, starting from the compound obtained in Example 3. The title product is obtained in the form of a greenish powder.

Melting point: 88-90° C.

### EXAMPLE 5

### Ethyl 3-{4-[2-(6-[cyclopropyl(methoxyimino)methyl]-2-oxo-1,3-benzothiazol-3(2H)-yl)ethoxy]phenyl}-2-(2,2,2-trifluoroethoxy)-propanoate

The procedure is as in Example 3, with the replacement of hydroxylamine hydrochloride with O-methylhydroxylamine hydrochloride. The title product is obtained in the form of an oil.

### Elemental Microanalysis:

| | C % | H % | N % |
|---|---|---|---|
| Calculated: | 57.24 | 5.16 | 4.94 |
| Found: | 57.34 | 5.38 | 4.85 |

### EXAMPLE 6

### 3-{4-[2-(6-[Cyclopropyl(methoxyimino)methyl]-2-oxo-1,3-benzothiazol-3(2H)-yl)ethoxy]phenyl}-2-(2,2,2-trifluoroethoxy)propanoic acid

The procedure is as in Example 2, starting from the compound obtained in Example 5. The title product is obtained in the form of a yellow powder.

Melting point: 33-34° C.

### Elemental Microanalysis:

| | C % | H % | N % |
|---|---|---|---|
| Calculated: | 55.76 | 4.68 | 5.20 |
| Found: | 55.93 | 5.04 | 4.84 |

### EXAMPLE 7

### (S)-3-{4-[2-(6-(Cyclopropylcarbonyl)-2-oxo-1,3-benzothiazol-3(2H)-yl)ethoxy]phenyl}-2-(2,2,2-trifluoroethoxy)propanoic acid

### Step A: (2S)-3-{4-[2-(6-(Cyclopropylcarbonyl)-2-oxo-1,3-benzothiazol-3(2H)-yl)ethoxy]phenyl}-N-[(1S)-2-hydroxy-1-phenylethyl]-2-(2,2,2-trifluoro-ethoxy)propanamide

The compound obtained in Example 2 (1 g) is dissolved in 90 ml of anhydrous dichloro-methane, and 330 μl of triethylamine are added. The temperature is lowered to 0° C. and 320 mg of HOBt then 450 mg of EDCI are added. After stirring for 15 minutes, 290 mg of (S)-phenylglycinol are added. The mixture is stirred for 20 hours at ambient temperature. The mixture is hydrolysed and then extracted with dichloromethane. The organic phases are dried and evaporated and the residue is purified on a semi-preparative column (eluant petroleum ether/ethyl acetate 35/65) to yield the title product in the form of a white solid.

Melting point: 198-202° C.

### Step 1B: (S)-3-{4-[2-(6-(Cyclopropylcarbonyl)-2-oxo-1,3-benzothiazol-3(2H)-yl)ethoxy]phenyl}-2-(2,2,2-trifluoroethoxy)propanoic acid

1.5 g of the compound obtained in Step A are dissolved in 60 ml of dioxane and then 60 ml of water are added. Subsequently, 52.3 ml of 4.6M sulphuric acid are added and the mixture is heated with dioxane, at reflux, for 4 hours. The mixture is hydrolysed in a large volume of water and extracted with dichloromethane. The organic phases are dried and evaporated and the residue is purified by chromatography on silica gel (eluant: dichloromethane then dichloromethane/methanol 98/2) to yield the title product in the form of a white solid.

Melting point: 126-130° C.

### EXAMPLE 8

### (R)-3-{4-[2-(6-(Cyclopropylcarbonyl)-2-oxo-1,3-benzothiazol-3(2H)-yl)ethoxy]phenyl}-2-(2,2,2-trifluoroethoxy)propanoic acid

### Step A: (2R)-3-{4-[2-(6-(Cyclopropylcarbonyl)-2-oxo-1,3-benzothiazol-3(2H)-yl)ethoxy]phenyl}-N-[(1S)-2-hydroxy-1-phenylethyl]-2-(2,2,2-trifluoro-ethoxy)propanamide

The procedure is as in Step A of Example 7, with isolation of the other diastereoisomer at the end of the semi-preparative chromatography.

Melting point: 150-154° C.

### Step B: (R)-3-{4-[2-(6-(Cyclopropylcarbonyl)-2-oxo-1,3-benzothiazol-3(2H)-yl)-ethoxy]phenyl}-2-(2,2,2-trifluoroethoxy)propanoic acid

The procedure is as in Step B of Example 7, starting from the compound obtained in Step A.

### EXAMPLE 9

### Sodium (S)-3-{4-[2-(6-(cyclopropylcarbonyl)-2-oxo-1,3-benzothiazol-3(2H)-yl)ethoxy]phenyl}-2-(2,2,2-trifluoroethoxy)propanoate

3.88 ml of 0.1N sodium hydroxide solution are added to 200 mg of the compound obtained in Example 7. The solution is stirred for 4 hours at ambient temperature and then the water is evaporated off by lyophilisation to yield the title product in the form of a white solid.

### Elemental Microanalysis:

| | C % | H % | N % | S % |
|---|---|---|---|---|
| Calculated: | 54.24 | 3.98 | 2.64 | 6.03 |
| Found: | 54.28 | 4.67 | 2.50 | 5.17 |

### EXAMPLE 10

### Sodium (R)-3-{4-[2-(6-(cyclopropylcarbonyl)-2-oxo-1,3-benzothiazol-3(2H)-yl)ethoxy]phenyl}-2-(2,2,2-trifluoroethoxy)propanoate

The procedure is as in Example 9, starting from the compound obtained in Example 8.

### Freeze-Dried Product

### Elemental Microanalysis:

| | C % | H % | N % |
|---|---|---|---|
| Calculated: | 54.24 | 3.98 | 2.64 |
| Found: | 53.61 | 4.92 | 2.77 |

### EXAMPLE 11

### Potassium (S)-3-{4-[2-(6-(cyclopropylcarbonyl)-2-oxo-1,3-benzo-thiazol-3(2H)-yl)ethoxy]phenyl}-2-(2,2,2-trifluoroethoxy)propanoate

The procedure is as in Example 9, with the replacement of sodium hydroxide solution with potassium hydroxide solution.

### Freeze-Dried Product.

### EXAMPLE 12

### Potassium (R)-3-{4-[2-(6-(cyclopropylcarbonyl)-2-oxo-1,3-benzo-thiazol-3(2H)-yl)ethoxy]phenyl}-2-(2,2,2-trifluoroethoxy)propanoate

The procedure is as in Example 9, starting from the compound obtained in Example 8 and with the replacement of sodium hydroxide solution with potassium hydroxide solution.

### Freeze-Dried Product.

### PHARMACOLOGICAL STUDY

### Example A

### Acute Toxicity Study

The acute toxicity was evaluated after oral administration to groups each comprising 8 mice (26±2 grams). The animals were observed at regular intervals during the course of the first day, and daily for two weeks following the treatment. The LD₅₀ (dose that causes the death of 50% of the animals) was evaluated and demonstrated the low toxicity of the compounds of the invention.

### Example B

### Effectiveness in Genetic Models

Mutations in laboratory animals and also different sensitivities to dietary regimens have allowed the development of animal models having non-insulin-dependent diabetes and hyperlipidaemia associated with obesity and with resistance to insulin. Genetic mice models (ob/ob) (Diabetes, 1982, 31 (1), 1-6) and Zucker (fa/fa) rats have been developed by various laboratories in order to understand the physiopathology of those diseases and test the effectiveness of new antidiabetic compounds (Diabetes, 1983, 32, 830-838).

Antidiabetic and Hypolipaemic Effect in the ob/ob Mouse

The 10-week-old male ob/ob mouse (Harlan) is used for the in vivo tests. The animals are kept in a light-darkness cycle of 12 hours at 25° C. This mouse has a basal hyperglycaemia of 2 g/l. The animals are randomly selected with regard to their glycaemia to form groups of eight. The compounds tested by the oral route are dissolved in a mixture of hydroxyethyl cellulose (HEC 1%) to be administered at 3 mg/kg in a volume of 10 ml/kg once per day for four days. The control group receives the solvents under the same conditions as the treated groups. The activity of the products is evaluated by measuring glycaemia, insulinaemia and triglyceridaemia 24 hours after the final administration and by measuring body weight daily.

The compounds of the invention demonstrate a very good capacity to lower glycaemia that is comparable to the effects obtained with rosiglitazone, which is used as reference substance.

By way of example, administered at a dose of 3 mg/kg to the ob/ob mouse by the oral route, the compound of Example 2 normalises glycaemia, insulinaemia and triglyceridaemia: respective reductions of 42%, 71% and 45% of the values observed in the control mice Administered at a dose of 1 mg/kg, the compound of Example 9 exhibits a 39% reduction in glycaemia, a 41% reduction in insulinaemia and a 52% reduction in triglyceridaemia. In addition, a 14% reduction in the weight gain observed in the ob/ob control mice is observed.

Furthermore, no side effect was observed during the in vivo tests.

### Example C

### Pharmaceutical Composition

| | |
|---|---|
| 1000 tablets each containing a dose of 5 mg of sodium | 5 g |
| (S)-3-4{4-[2-(6-(cycloproplycarbonyl)-2-oxo-1,3-benzothiazol- | |
| 3(2H)-yl)ethoxyl]phenyl}-2-(2,2,2-trifluoroethoxy) | |
| propanoate (Example 9) | |
| wheat starch | 20 g |
| maize starch | 20 g |
| lactose | 30 g |
| magnesium stearate | 2 g |
| silica | 1 g |
| hydroxypropyl cellulose | 2 g |

### Example D

### Variation in Body Weight

Male C57 Black 6 ob/ob mice 8 to 12 weeks old were used. After being placed in quarantine for one week, they were weighed and then randomly selected as a function of their weight, and 6 homogeneous groups (starting weight not significantly different) were formed. After weighing the animals, the different associations according to the invention between a compound of formula (I) and an antioxidant agent that are to be tested are injected intraperitoneally once per day for 7 days. The molecules are injected in a 5% DMSO/15% Solutol/q.s. H₂O solution heated to 65° C. to ensure good dissolution. The solution is in addition preheated prior to injection. The mice are weighed every day and the weight attained after 7 days of treatment is recorded.

The results obtained clearly demonstrate:
that the association according to the invention between a compound of formula (I) and an antioxidant agent allows a significant reduction in the weight of the obese mice to be achieved,
that there is a synergy between the 2 components of the association, the loss in weight ascertained being far greater using the association than when using each component administered on its own.

### Example E

### Pharmaceutical Composition

| | |
|---|---|
| 100 tablets each containing a dose of 30 mg of sodium | 3 g |
| (S)-3-{4-[2-(6-(cyclopropylcarbonyl)-2-oxo-1,3- | |
| benzothiazol-3(2H)-yl)ethoxy]phenyl}-2- | |
| (2,2,2-trifluoroethoxy)propanoate (Example 9) | |
| and 10 mg of coenzyme Q₁₀ | |
| coenzyme Q₁₀ | 1 g |
| wheat starch | 20 g |
| maize starch | 20 g |
| lactose | 30 g |
| magnesium stearate | 2 g |
| silica | 1 g |
| hydroxypropyl cellulose | 2 g |

1-26. (canceled)

27. A compound selected from those of formula (I): 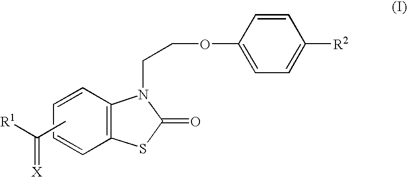
wherein:
R¹ represents a (C₃-C₈)cycloalkyl group,
R² represents a group of formula (II): 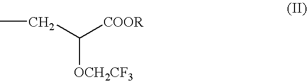
wherein R represents a hydrogen atom or a linear or branched (C₁-C₆)alkyl group,
X represents an oxygen atom or an N—OR′ group wherein R′ represents a hydrogen atom, a linear or branched (C₁-C₆)alkyl group, an aryl group or an aryl-(C₁-C₆)alkyl group in which the alkyl moiety may be linear or branched,
its geometric isomers, enantiomers and diastereoisomers and pharmaceutically acceptable addition salts thereof with an acid or a base,
wherein:
“geometric isomers” means that, when X represents an N—OR′ group, the oxime R¹—C(═N—OR′)— may have the Z or E configuration,
“aryl” means a phenyl or naphthyl group, wherein each of these groups may optionally be substituted by from 1 to 3 groups selected from linear or branched (C₁-C₆)alkyl, linear or branched (C₁-C₆)polyhaloalkyl, linear or branched (C₁-C₆)alkoxy, hydroxy, carboxy, formyl, amino (optionally substituted by one or two linear or branched (C₁-C₆)alkyl groups), ester, amido, nitro, cyano, and halogen.

28. The compound of claim 27, wherein R¹ represents a cyclopropyl group.

29. The compound of claim 27, wherein R represents a hydrogen atom or an ethyl group.

30. The compound of claim 27, wherein R² represents the group —CH₂—CH(OCH₂CF₃)(COOH).

31. The compound of claim 27, wherein R² has the R or S configuration.

32. The compound of claim 27, wherein X represents an oxygen atom.

33. The compound of claim 27, which is selected from 3-{4-[2-(6-(cyclopropyl-carbonyl)-2-oxo-1,3-benzothiazol-3(2H)-yl)ethoxy]phenyl}-2-(2,2,2-trifluoroethoxy)-propanoic acid, its geometric isomers, enantiomers and diastereoisomers and its pharmaceutically acceptable addition salts with an acid or a base.

34. The compound of claim 27, which is selected from (S)-3-{4-[2-(6-(cyclopropyl-carbonyl)-2-oxo-1,3-benzothiazol-3(2H)-yl)ethoxy]phenyl}-2-(2,2,2-trifluoroethoxy)-propanoic acid and its pharmaceutically acceptable addition salts with a base.

35. The compound of claim 27, which is sodium (S)-3-{4-[2-(6-(cyclo-propylcarbonyl)-2-oxo-1,3-benzothiazol-3(2H)-yl)ethoxy]phenyl}-2-(2,2,2-trifluoro-ethoxy)propanoate.

36. A pharmaceutical composition comprising as active ingredient at least one compound of claim 27, alone or in combination with one or more pharmaceutically acceptable excipients.

37. A method for treating a condition selected from hyperglycaemia, dyslipidaemia non-insulin-dependent type II diabetes, insulin resistance, glucose intolerance, disorders associated with syndrome X, coronary artery disease, cardiovascular diseases, renal disorders, retinopathy, disorders associated with the activation of endothelial cells, psoriasis, polycystic ovary syndrome, dementia, osteoporosis, intestinal inflammatory disorders, myotonic dystrophy, pancreatitis, arteriosclerosis, xanthoma, type I diabetes, obesity, regulation of appetite, anorexia, bulimia, anorexia nervosa, cancer pathologies, including hormone-dependent cancers, breast cancer, and colon cancer, and conditions requiring an angiogenesis inhibitor, comprising the step of administering to a living animal body, including a human, a therapeutically effective amount of a compound of claim 27.

38. A composition comprising a compound selected from those of formula (I): 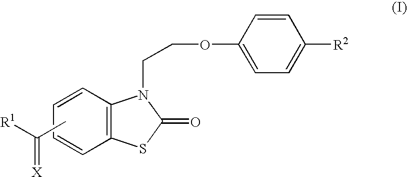
wherein
R¹ represents a (C₃-C₈)cycloalkyl group,
R² represents a group of formula (II): 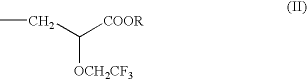
wherein R represents a hydrogen atom or a linear or branched (C₁-C₆)alkyl group,
X represents an oxygen atom or an N—OR′ group wherein R′ represents a hydrogen atom, a linear or branched (C₁-C₆)alkyl group, an aryl group or an aryl-(C₁-C₆)alkyl group in which the alkyl moiety may be linear or branched,
its geometric isomers, enantiomers and diastereoisomers and pharmaceutically acceptable addition salts thereof with an acid or a base,
wherein:
“geometric isomers” means that, when X represents an N—OR′ group, the oxime R¹—C(═N—OR′)— may have the Z or E configuration,
“aryl” means a phenyl or naphthyl group, wherein each of these groups may optionally be substituted by from 1 to 3 groups selected from linear or branched (C₁-C₆)alkyl, linear or branched (C₁-C₆)polyhaloalkyl, linear or branched (C₁-C₆)alkoxy, hydroxy, carboxy, formyl, amino (optionally substituted by one or two linear or branched (C₁-C₆)alkyl groups), ester, amido, nitro, cyano, and halogen,
and an antioxidant agent.

39. The composition of claim 38, wherein the compound of formula (I) is selected from 3-{4-[2-(6-(cyclopropylcarbonyl)-2-oxo-1,3-benzothiazol-3(2H)-yl)ethoxy]phenyl}-2-(2,2,2-trifluoroethoxy)propanoic acid, its enantiomers and diastereoisomers and its pharmaceutically acceptable addition salts with an acid or a base.

40. The composition of claim 38, wherein the antioxidant agent is coenzyme Q₁₀.

41. A pharmaceutical composition comprising as active ingredient a composition of claim 38, together with one or more pharmaceutically acceptable excipients.

42. A method for treating obesity, comprising the step of administering to a living animal body, including a human, a therapeutically effective amount of a composition of claim 38.

43. A method for treating obesity induced by therapeutic treatment, comprising the step of administering to a living animal body, including a human, a therapeutically effective amount of a composition of claim 38.

44. A method for treating obesity induced by treatment of type I or type II diabetes, comprising the step of administering to a living animal body, including a human, a therapeutically effective amount of a composition of claim 38.

45. A method for treating overweight characterised by a body mass index greater than 25 and less than 30, comprising the step of administering to a living animal body, including a human, a therapeutically effective amount of a composition of claim 38.

46. A method for treating overweight characterised by a body mass index greater than 25 and less than 30 induced by therapeutic treatment, comprising the step of administering to a living animal body, including a human, a therapeutically effective amount of a composition of claim 38.

47. A method for treating overweight characterised by a body mass index greater than 25 and less than 30 induced by treatment of type I or type II diabetes, comprising the step of administering to a living animal body, including a human, a therapeutically effective amount of a composition of claim 38.
